(19) United States
(12) Patent Application Publication
(10) Pub. No.: US 2010/0256736 A1
(43) Pub. Date: Oct. 7, 2010

(54) COVERED BALLOON EXPANDABLE STENT DESIGN AND METHODOF COVERING

(75) Inventors: JAMES D. PURDY, Lafayette, IN (US); Richard A. Swift, South Bend, IN (US); Blayne A. Roeder, Lafayette, IN (US); Alan R. Leewood, Lafayette, IN (US); Jichao Sun, West Lafayette, IN (US)

Correspondence Address: BRINKS HOFER GILSON & LIONE/CHICAGO/COOK, PO BOX 10395, CHICAGO, IL, 60610, US

(73) Assignee: MED Institute, Inc.

(21) Appl. No.: 12/817,845

(22) Filed: Jun. 17, 2010

Related U.S. Application Data
(62) Division of application No. 12/022,390, filed on Jan. 30, 2008.
(60) Provisional application No. 60/898,897, filed on Feb. 1, 2007.

(51) Int. Cl. A61F 2/82; B05D 3/12

(52) U.S. Cl. 623/1.15; 623/1.44; 427/2.25

(57) ABSTRACT

A balloon expandable covered stent consists of a plurality of primary stent units, each having an undulating shape defined by a series of primary strut members converging to form peaks and valleys. The primary stent units are assembled into a single cylindrical structure of the stent by connecting corresponding peaks with secondary strut members. Generally, surfaces of the stent may then coated with a polymeric, hyper-elastic material, preferably Thoralon®, by pre-expanding the stent prior to coating.

### RELATED APPLICATIONS

This application claims priority to U.S. Provisional Application No. 60/898,897, filed on Feb. 1, 2007, entitled “Covered Balloon Expandable Stent Design and Method of Covering.”

### BACKGROUND OF THE INVENTION

This invention relates generally to the field of stents for use primarily in ducts and vessels of the body, and more particularly, to the area of covered expandable stents which expand after implantation in the body.

Stent-grafts have proven to be an effective medical device for minimally invasive treatment of vascular occlusions such as atherosclerosis and restenosis. Stent-grafts are typically shaped as hollow cylindrical structures and constructed of a metal stent with at least one non-metal coating on the stent.

Various stent designs are known in the art. These stents form vascular prostheses fabricated from biocompatible materials. Stents are typically used to expand and maintain patency of hollow vessels, such as blood vessels or other body orifices.

Known stent designs, however, do not have the strength or uniform expansion needed to allow hyperelastic coating material to effectively encapsulate an expandable stent. The ability to expand hyperelastic covering material in a relatively uniform fashion during the expansion of the stent and allowing sufficient room between the stentinterstitials is an object of this invention.

### SUMMARY OF THE INVENTION

The stent device described below may overcome the aforementioned problems and relates to a medical device, and more particularly, to a covered stent and method of making the same that has 151015202530 the strength and uniform expansion needed to allow hyperelastic coating material effectively to encapsulate an expandable stent.

One embodiment includes a covered stent for insertion into a vessel of a patient, the stent including a polymeric outer covering and a tubular member having a distal end, a proximal end, and a longitudinal axis extending therebetween. The tubular member is defined by a plurality of primary stent units extending between the proximal and distal ends, the primary stent units comprising a plurality of primary strut members defining an undulating wave pattern of repeating u-shaped peaks and valleys. The peaks and valleys have a radius of curvature that is constant throughout the u-shape. The tubular member further includes a plurality of secondary strut members connecting adjacent primary stent units to one another, the secondary strut members extending from an inner surface of a first ushaped peak to an outer surface of a second u-shaped peak.

The covered stent as described above, wherein the primary strut members are tapered.

The covered stent as described above, wherein the polymeric outer coating comprises Thoralon®.

The covered stent as described above, wherein the polymericout coating comprises at least three layers, at least one of the layers comprising a non-porous material.

The covered stent as described above, wherein the stent comprises at least four primary stent units, the primary stent units being aligned parallel to one another.

The covered stent as described above, wherein the stent is made of stainless steel.

The covered stent as described above, wherein the stent is made of cobalt-chromium steel.

Another embodiment includes a method for coating a stent for insertion into a vessel of a patient, including the steps of expanding the stent between 75% and 95% of its maximum diameter, inserting a mandrel through a lumen of the stent, coating the stent with a polymeric coating, drying the polymeric coating while the stent is expanded, removing the mandrel, and crimping the stent over a delivery device.

The method as described above, wherein the polymeric coating comprises Thoralon®.

The method as described above, wherein the polymeric coating comprises at least three layers, at least one of which comprises Thoralon®.

The method as described above, wherein the stent includes a tubular member having a distal end, a proximal end, and a longitudinal axis extending therebetween. The tubular member is defined by a plurality of primary stent units extending between the proximal and distal ends, the primary stent units comprising a plurality of primary strut members defining an undulating wave pattern of repeating u-shaped peaks and valleys. The peaks and valleys have a radius of curvature that is constant throughout the u-shape. The tubular member further includes a plurality of secondary strut members connecting adjacent primary stent units to one another. The struts extend from an inner surface of a first u-shaped peak to an outer surface of a second u-shaped peak.

The method as described above, wherein the stent is expanded to approximately 85% of said maximum diameter prior to the stent being coated with the polymeric coating material.

The method as described above, wherein the coating material comprises at least a first porous layer, a non-porous layer, and a second porous layer, wherein the first and second porous layers encase the nonporous layer.

### BRIEF DESCRIPTION OF THE DRAWINGS

FIG. 1 is a side view of one embodiment of a stent in a collapsed configuration;

FIG. 2 is a schematic representation of the taper formed on a strut member;

FIG. 3 is a top view of a flat section of the stent of FIG. 1 in a collapsed configuration;

FIG. 4 is a side view of a stent in an expanded configuration;

FIG. 5 is an end view of a coated stent; and

FIG. 6 is an end view of a coated stent.

### DETAILED DESCRIPTION OF THE PREFERRED EMBODIMENTS

A balloon expandable covered stent consists of a plurality of primary stent units, each having an undulating shape defined by a series of primary strut members converging to form peaks and valleys. The primary stent units are generally expandable in a circumferential direction. The primary stent units are assembled into a single cylindrical structure of the stent by connecting corresponding peaks with secondary strut members. The stent units are connected to one another by the secondary strut members until the desired length of the stent is acquired. All surfaces of the stent are then coated with a polymeric, hyper-elastic material, preferably Thoralon®. The stent may be coated according to the method described in this application or any other suitable method.

The stent is tailored to meet the needs of an iliac branch vessel deployment procedure and may be constructed from 601 stainless steel, L605 Cobalt-Chromium steel, or other suitable material. The stent has a unique strut configuration to accommodate the needs of a hyperelastic covering material, such as Thoralon®. Although the stent is primarily suited for use in the iliac branch vessel, it may also be suitable for other applications.

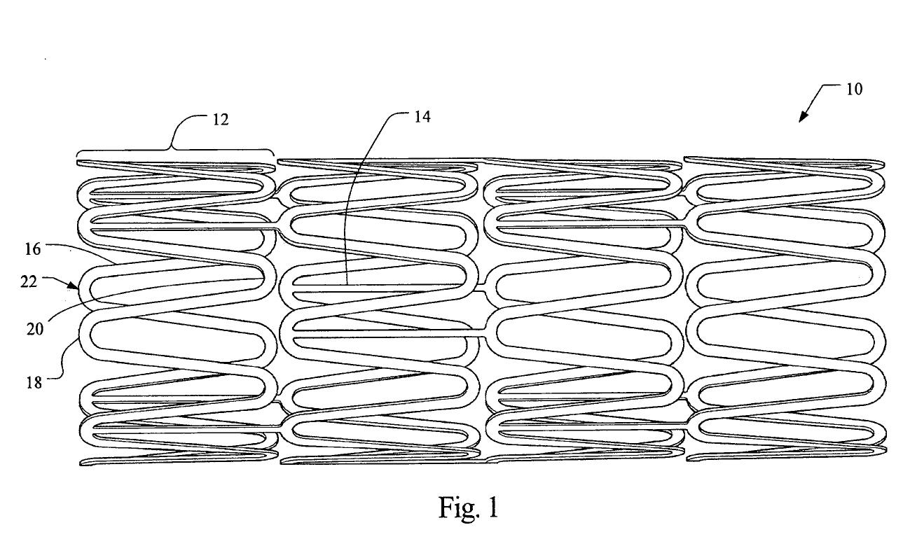

Referring now to FIG. 1, stent 10 may include a plurality of primary stent units 12 connected to one another with secondary strut members 14. The primary stent units 12 may consist of a plurality of primary strut members 16 converging to form peaks 18 and valleys 20, arranged in a circular undulating pattern so that the radial curvature of each peak 18 and valley 20 is consistent throughout each u-shaped curve 22. In addition to creating a uniform radius of curvature at the peaks 18 and valleys 20, the primary strut members 16 are tapered to improve the stiffness distribution, allowing for a significantly improved strain distribution when expanded.

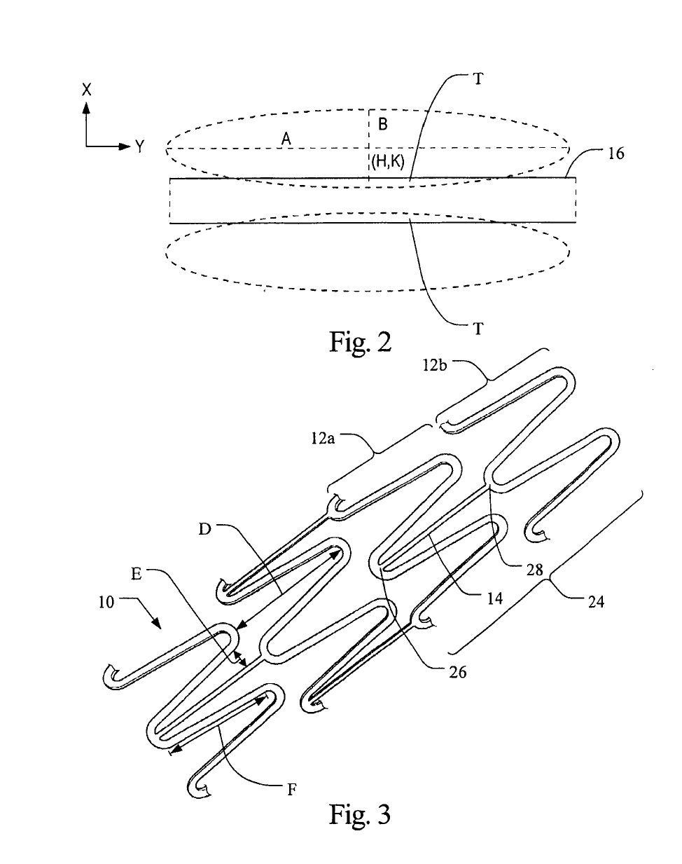

Referring to FIG. 2, the primary strut member 16 may be tapered in accordance with the equation:

(X_{—}H)2/A2+(y_{—}K)2/B2=1,

Where x and yare the length and width of the primary strut 16, respectively, and A is the semimajor diameter and B is equal to the semiminor diameter of the ellipse. Typical values for the above equation would generally be A=0.91 mm and B=0.016 mm, with a maximum strut width reduction in the taper zone T of about 30%.

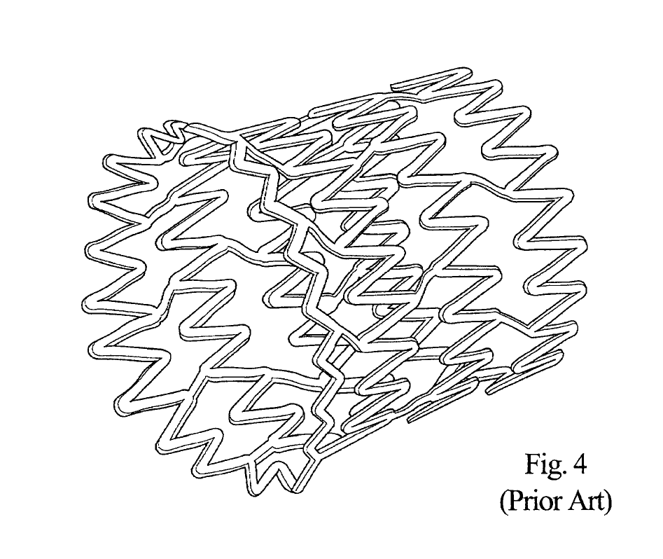

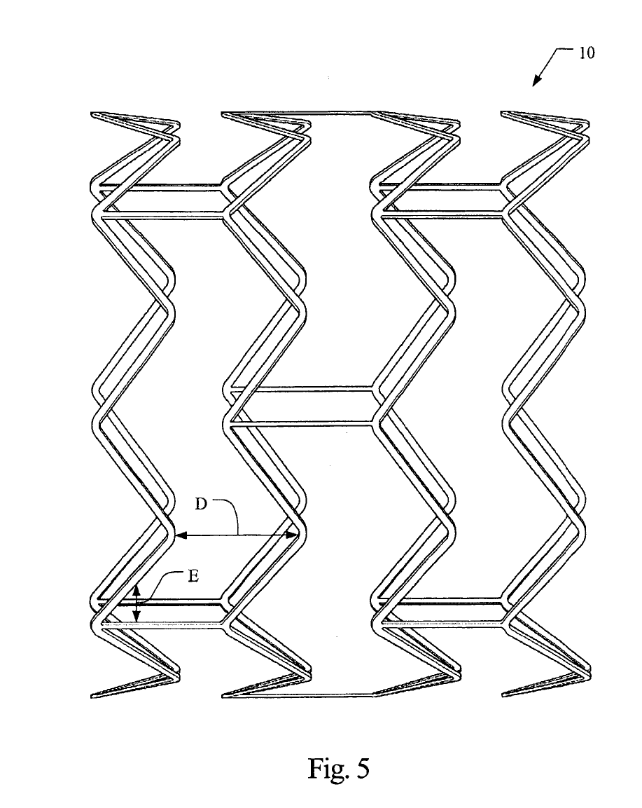

In order to produce a desired balloon expandable stent 10, a solid cannula may be laser cut according to the desired configuration. Generally, using the primary stent units 12, two or more primary stent units 12, having the same shape, are arranged parallel to one another. The primary stent units 12 are connected to one another using a plurality of secondary struts 14. The secondary struts 14 connect respective distal surfaces 26 of the peaks 18 of the first primary stent unit 12a with the corresponding proximal surfaces 28 of the peaks 18 of the second primary stent unit 12b, as shown in FIG. 3. The peaks are generally u-shaped with a constant radius of curvature, as shown in FIG. 3, as opposed to a v shape, shown in FIG. 4, or other shape which would not allow space for the coating material to compress as the stent is crimped.

FIG. 3 depicts a flat view of a portion stent 10 cut from a cylindrical piece of the stent of FIG. 1. Referring now to FIG. 3, the relationship between the peaks 18 of the respective primary units 12 is generally linear along a 1800 angle, as illustrated by dimension D. Dimension D also represents the relative distance between the peaks 18, which remains constant when the stent 10 is expanded and collapsed. Angle E is defined by the angle between the primary strut member 16 and the secondary strut member 14. This angle increases and decreases uniformly allowing uniform expansion and crimping of the coating material. The uniform expansion and compression of this angle induces a relatively uniform expansion of the Thoralon® covering and minimizes regions of high tensile strain. Regions of compressive strain are also minimized during expansion, preventing wrinkling of the Thoralon® when expanded to the nominal stent diameter. Upon expansion of the stent 10, the trapezoidal shape formed by angle E and dimension D will generally expand to allow the coating material to expand uniformly, as shown in FIGS. 3 and 5, respectively. Furthermore, the length of the primary strut member 16 may be increased or decreased, depending on the desired outer diameter of the stent 10, keeping all other variables constant. Generally, one embodiment of the stent 10 may have an outer diameter of approximately 5 mm to approximately 8 mm. A stent of this diameter would generally be suitable for delivery using a 7 French catheter. Another embodiment of stent 10 would include an outer diameter of approximately 9 mm to approximately 12 mm and would be suitable for delivery in an 8 French catheter. The primary difference between the two embodiments may be the length of the primary strut member.

The stent 10 of the present invention may be coated with a hyper-elastic material, preferably a biocompatible polyurethane. One example of a biocompatible polyurethane is THORALON (THORATEC, Pleasanton, Calif.). As described in U.S. Pat. Application Publication No. 2002/0065552A1 and U.S. Pat. No. 4,675,361, both of which are incorporated herein by reference. THORALON is a polyurethane base polymer (referred to as BPS-215) blended with a siloxane containing surface modifying additive (referred to as SMA-300). The concentration of the surface modifying additive may be in the range of 0.5% to 5% by weight of the base polymer.

The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference.

The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MOI) and ethylene diamine (ED).

THORALON can be manipulated to provide either porous or non-porous THORALON. Porous THORALON can be formed by mixing the polyetherurethane urea (BPS-215), the surface modifying additive (SMA-300) and a particulate substance in a solvent. The particulate may be any of a variety of different particulates or pore forming agents, including inorganic salts. Preferably the particulate is insoluble in the solvent. The solvent may include dimethyl formamide (OMF), tetrahydrofuran (THF), dimethyacetamide (OMAC), dimethyl sulfoxide (OMSO), or mixtures thereof. The composition can contain from about 5 wt % to about 40 wt % polymer, and different levels of polymer within the range can be used to fine tune the viscosity needed for a given process. The composition can contain less than 5 wt % polymer for some spray application embodiments. The particulates can be mixed into the composition. For example, the mixing can be performed with a spinning blade mixer for about an hour under ambient pressure and in a temperature range of about 18° C. to about 27° C. The entire composition can be cast as a sheet, or coated onto an article such as a mandrel or a mold. In one example, the composition can be dried to remove the solvent, and then the dried material can be soaked in distilled water to dissolve the particulates and leave pores in the material. In another example, the composition can be coagulated in a bath of distilled water. Since the polymer is insoluble in the water, it will rapidly solidify, trapping some or all of the particulates. The particulates can then dissolve from the polymer, leaving pores in the material. It may be desirable to use warm water for the extraction, for example water at a temperature of about 60° C. The resulting pore diameter can also be substantially equal to the diameter of the salt grains.

The porous polymeric sheet can have a void-to-volume ratio from about 0.40 to about 0.90. Preferably the void-to-volume ratio is from about 0.65 to about 0.80. The resulting void-to-volume ratio can be substantially equal to the ratio of salt volume to the volume of the polymer plus the salt. Void-to-volume ratio is defined as the volume of the pores divided by the total volume of the polymeric layer including the volume of the pores. The void-to-volume ratio can be measured using the protocol described in AAMI (Association for the Advancement of Medical Instrumentation) VP20-1994, Cardiovascular Implants—Vascular Prosthesis section 8.2.1.2, Method for Gravimetric Determination of Porosity. The pores in the polymer can have an average pore diameter from about 1 micron to about 400 microns. Preferably the average pore diameter is from about 1 micron to about 100 microns, and more preferably is from about 1 micron to about 10 microns. The average pore diameter is measured based on images from a scanning electron microscope (SEM). Formation of porous THORALON is described, for example, in U.S. Pat. No. 6,752,826 and 2003/0149471 A1, both of which are incorporated herein by reference.

Non-porous THORALON can be formed by mixing the polyetherurethane urea (BPS-215) and the surface modifying additive (SMA-300) in a solvent, such as dimethyl formamide (DMF), tetrahydrofuran (THF), dimethyacetamide (DMAC), dimethyl sulfoxide (DMSO). The composition can contain from about 5 wt % to about 40 wt % polymer, and different levels of polymer within the range can be used to fine tune the viscosity needed for a given process. The composition can contain less than 5 wt % polymer for some spray application embodiments. The entire composition can be cast as a sheet, or coated onto an article such as a mandrel or a mold. In one example, the composition can be dried to remove the solvent.

THORALON has been used in certain vascular applications and is characterized by thromboresistance, high tensile strength, low water absorption, low critical surface tension, and good flex life. THORALON is believed to be biostable and to be useful in vivo in long term blood contacting applications requiring biostability and leak resistance. Because of its flexibility, THORALON is useful in larger vessels, such as the abdominal aorta, where elasticity and compliance is beneficial.

A variety of other biocompatible polyurethanes may also be employed. These include polyurethane that preferably include a soft segment and include a hard segment formed from a diisocyanate and diamine. For example, polyurethane with soft segments such as PTMO, polyethylene oxide, polypropylene oxide, polycarbonate, polyolefin, polysiloxane (Le. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole.

The diisocyanate used as a component of the hard segment may be represented by the formula OCN—R—NCO, where —R— may be aliphatic, aromatic, cycloaliphatic or a mixture of aliphatic and aromatic moieties. Examples of diisocyanates include MOI, tetra methylene diisocyanate, hexamethylene diisocyanate, trimethyhexamethylene diisocyanate, tetramethylxylylene diisocyanate, 4,4′-dicyclohexylmethane diisocyanate, dimer acid diisocyanate, isophorone diisocyanate, metaxylene diisocyanate, diethylbenzene diisocyanate, decamethylene 1,10 diisocyanate, cyclohexylene 1,2-diisocyanate, 2,4-toluene diisocyanate, 2,6-toluene diisocyanate, xylene diisocyanate, m-phenylene diisocyanate, hexahydrotolylene diisocyanate (and isomers), naphthylene 1,5-diisocyanate, 1-methoxyphenyl 2,4-diisocyanate, 4,4′-biphenylene diisocyanate, 3,3′-dimethoxy-4,4′-biphenyl diisocyanate and mixtures thereof.

The diamine used as a component of the hard segment includes aliphatic amines, aromatic amines and amines containing both aliphatic and aromatic moieties. For example, diamines include ethylene diamine, propane diamines, butanediamines, hexanediamines, pentane diamines, heptane diamines, octane diamines, m-xylylene diamine, 1,4-cyclohexane diamine, 2-methypentamethylene diamine, 4,4′-methylene dianiline, and mixtures thereof. The amines may also contain oxygen and/or halogen atoms in their structures.

Other applicable biocompatible polyurethanes include those using a polyol as a component of the hard segment. Polyols may be aliphatic, aromatic, cycloaliphatic or may contain a mixture of aliphatic and aromatic moieties. For example, the polyol may be ethylene glycol, diethylene glycol, triethylene glycol, 1,4-butanediol, 1,6-hexanediol, 1,8octanediol, propylene glycols, 2,3-butylene glycol, dipropylene glycol, dibutylene glycol, glycerol, or mixtures thereof.

Biocompatible polyurethanes modified with cationic, anionic and aliphatic side chains may also be used. See, for example, U.S. Pat. No. 5,017,664.

Other biocompatible polyurethanes include: segmented polyurethanes, such as BIOSPAN; polycarbonate urethanes, such as BIONATE; and polyetherurethanes, such as ELASTHANE; (all available from POLYMER TECHNOLOGY GROUP, Berkeley, Calif.).

Other biocompatible polyurethanes include polyurethanes having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxanepolyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL -AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which POMS is incorporated into the polymer soft segment with PTMO (PURSIL) or an aliphatic hydroxy-terminated polycarbonate (CARBOSIL). The hard segment consists of the reaction product of an aromatic diisocyanate, MOI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference.

In addition, any of these biocompatible polyurethanes may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference.

In order to coat stent 10 with THORALON, the stent may be first pre-expanded to between 75% and 95% of its maximum diameter. Generally, the stent will be expanded to 85% of its maximum diameter. The stent is then placed on a glass mandrel, in expanded condition. By pre-expanding the stent, the THORALON is prevented from undergoing undue strain from an additional expansion before the stent's actual use.

Once on the glass mandrel, the balloon expandable stent is dipped in the coating materials 30 and dried at approximately 40° to 60° C. for approximately 90 minutes. The stent 10 is coated using known THORALON coating techniques, as disclosed in U.S. Pat. No. 6,752,826, the disclosure of which is incorporated herein. After drying, the stent 10 is removed from the mandrel and the excess coating material 30 is trimmed using known techniques. The stent 10 is then crimped over a standard balloon catheter with a desired outer diameter.

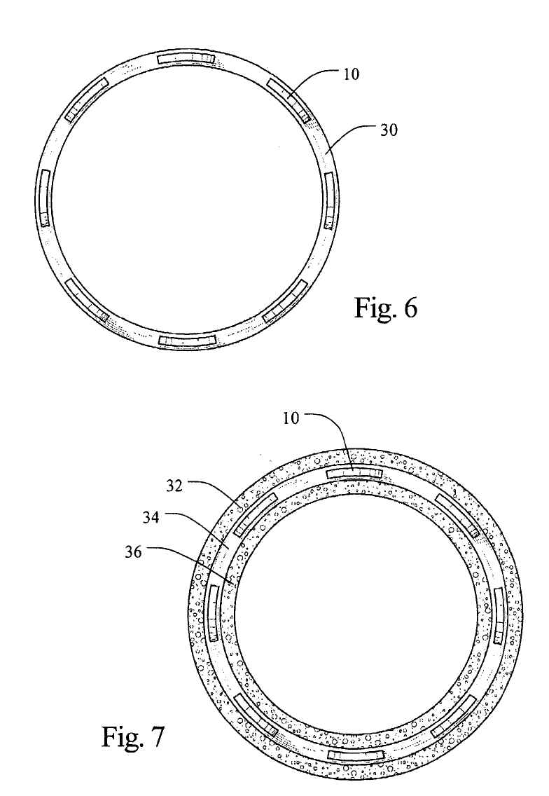

The surfaces of the stent 10 may be completely coated with coating material 30, as shown in FIG. 6. The resulting stent graft can include a porous outer layer 32, a non-porous middle layer 34, and a porous luminal layer 36. The non-porous middle layer 34 may contain the stent 10, as described above, or any other suitable stent structure, as shown in FIG. 7.

The stent 10 is particularly suitable for use with a material such as THORALON because when a coated stent is crimped, excess material gathers between the interstices of the stent 10. Stent 10 provides an open area that, when crimped onto a delivery device, allows for the extra material to collect, as shown in FIG. 3.

Although the invention has been shown and described with respect to preferred embodiments, alterations and modification of the components and methods of the invention may occur to those skilled in the art upon reading and understanding this specification. Accordingly, the present invention is defined by the scope of the claims below and not by the description provided above.

1. A covered stent for insertion into a vessel of a patient, said stent comprising:
a polymeric outer covering;
a tubular member having a distal end, a proximal end, and a longitudinal axis extending therebetween, said tubular member defined by a plurality of primary stent units longitudinally spaced apart between said proximal and distal ends, said primary stent units comprising a plurality of primary strut members defining an undulating wave pattern of repeating ushaped peaks and valleys, wherein said peaks and valleys have a radius of curvature that is constant throughout said u-shape;
said tubular member further comprising a plurality of secondary strut members connecting adjacent primary stent units to one another, said struts extending from a distal surface of a first u-shaped peak to a proximal surface of a second u-shaped peak.

2. The covered stent of claim 1, wherein said primary strut members are tapered.

3. The covered stent of claim 2, wherein said polymeric outer coating comprises Thoralon®.

4. The covered stent of claim 2, wherein said polymeric outer coating comprises at least three layers, at least one of the layers comprising a non-porous material.

5. The covered stent of claim 2, wherein said stent comprises at least four primary stent units, said stent units being aligned parallel to one another.

6. The covered stent of claim 2, wherein said stent comprises stainless steel.

7. The covered stent of claim 2, wherein said stent comprises cobalt-chromium steel.

8. A method for coating a balloon expandable stent for insertion into a vessel of a patient, comprising the steps of:
expanding said stent between 75% and 95% of its maximum diameter; inserting a mandrel through a lumen of said stent;
coating said stent with a polymeric coating;
drying said polymeric coating while said stent is expanded;
removing said mandrel; and
crimping said stent over a delivery device.

9. The method of claim 8, wherein said polymeric coating comprises Thoralon®.

10. The method of claim 9, wherein said polymeric coating comprises at least three layers, at least one of which comprises Thoralon®.

11. The method of claim 10, wherein said stent comprises:
a tubular member having a distal end, a proximal end, and a longitudinal axis extending therebetween, said tubular member defined by a plurality of primary stent units longitudinally spaced apart between said proximal and distal ends, said primary stent units comprising a plurality of primary strut members defining an undulating wave pattern of repeating ushaped peaks and valleys, wherein said peaks and valleys have a radius of curvature that is constant throughout said u-shape;
said tubular member further comprising a plurality of secondary strut members connecting adjacent primary stent units to one another, said struts extending from a distal surface of a first u-shaped peak to a proximal surface of a second u-shaped peak.

12. The method of claim 9, wherein said stent is expanded to approximately 85% of said maximum diameter prior to said stent being coated with said polymeric coating material.

13. The method of claim 9, wherein said coating material comprises 5 at least a first porous layer, a non-porous layer, and a second porous layer, wherein said first and second porous layers encase said non-porous layer.
